SEQUENCE LISTING (64 sequences)

SEQ ID NO: 1 - protein, 21 aa, Homo sapiens
CSCSSLMDKECVYFCHLDIIW

SEQ ID NO: 2 - protein, 21 aa, Felis catus
CSCSSLLDKECVYFCHLDIIW

SEQ ID NO: 3 - protein, 21 aa, Oncorhynchus keta
CSCATFLDKECVYFCHLDIIW

SEQ ID NO: 4 - protein, 21 aa, Danio rerio
CSCASFLDKECVYFCHLDIIW

SEQ ID NO: 5 - protein, 21 aa, Gallus gallus
CSCSSLLDEECVYFCHLDIIW

SEQ ID NO: 6 - protein, 21 aa, Monodelphis domestica
CSCSSLLDKECVYFCLLDIIW

SEQ ID NO: 7 - protein, 21 aa, Homo sapiens
CSCSSWLDKECVYFCHLDIIW

SEQ ID NO: 8 - protein, 21 aa, Rattus norvegicus
CSCNSWLDKECVYFCHLDIIW

SEQ ID NO: 9 - protein, 21 aa, Gallus gallus
CSCNSWLDKECIYFCHLDIIW

SEQ ID NO: 10 - protein, 21 aa, Homo sapiens
CTCFTYKDKECVYYCHLDIIW

SEQ ID NO: 11 - protein, 21 aa, Monodelphis domestica
CTCYTYKDKECVYYCHLDIIW

SEQ ID NO: 12 - protein, 21 aa, Bos taurus
CTCFTYKDRECVYYCHLDIIW

SEQ ID NO: 13 - protein, 21 aa, Danio rerio
CTCYSYKDKECVYYCHLDIIW

SEQ ID NO: 14 - protein, 21 aa, Artificial
  Synthetic Endothelin-1 analog
ASASSLMDKEAVYFAHLDIIW

SEQ ID NO: 15 - protein, 21 aa, Atractaspis engadensisan
CSCKDMTDKECLNFCHQDVIW

SEQ ID NO: 16 - protein, 21 aa, Atractaspic engadensisan
CSCKDMTDKECLYFCHQDVIW

SEQ ID NO: 17 - protein, 21 aa, Atractaspic engadensisan
CTCNDMTDEECLNFCHQDVIW

SEQ ID NO: 18 - protein, 21 aa, Atractaspic engadensisan
CTCNDMTDKECLYFCHEDIIW

SEQ ID NO: 19 - protein, 14 aa, Artificial
  modified endothelin-1 fragment
  MOD_RES (1)..(1): succinyl
DEEAVYFAHLDIIW

SEQ ID NO: 20 - protein, 81 aa, Phyllomedusa bicolor
  MOD_RES (81)..(81): OPTIONAL AMIDATION
MAFLKKSLFLVLFLGLVSLSICEEEKRENEDEEEQEDDEQSEMKRGLWSKIKEVGKEAAKAAAKAAGKAALGAVSEAVGEQ

SEQ ID NO: 21 - protein, 78 aa, Phyllomedusa bicolor
  MOD_RES (78)..(78): OPTIONAL AMIDATION
MDILKKSLFLVLFLGLVSLSICEEEKRENEDEEKQDDEQSEMKRAMWKDVLKKIGTVALHAGKAALGAVADTISQGEQ

SEQ ID NO: 22 - protein, 77 aa, Phyllomedusa bicolor
  MOD_RES (77)..(77): OPTIONAL AMIDATION
MAFLKKSLFLVLFLGLVSLSICEEEKRENEDEEEQEDDEQSEMKRGLWSKIKEAGKAALTAAGKAALGAVSDAVGEQ

SEQ ID NO: 23 - protein, 76 aa, Phyllomedusa bicolor
  MOD_RES (76)..(76): OPTIONAL AMIDATION
MAFLKKSLFLVLFLGLVSLSICEEEKRENKDEIEQEDDEQSEEKRALWKDILKNVGKAAGKAVLNTVTDMVNQGEQ

SEQ ID NO: 24 - protein, 81 aa, Phyllomedusa bicolor
  MOD_RES (81)..(81): OPTIONAL AMIDATION
MASLKKSLFLVLFLGLVSLSICEEEKRENEDEEEQEDDEQSEMKRGLWSNIKTAGKEAAKAALKAAGKAALGAVTDAVGEQ

SEQ ID NO: 25 - protein, 72 aa, Phyllomedusa bicolor
  MOD_RES (72)..(72): OPTIONAL AMIDATION
MDILKKSLFLVLFLGLVSLSICEEEKRENEDEEKQDDEQSEMKRAMWKDVLKKIAGKAALGAVADTISQGEQ

SEQ ID NO: 26 - protein, 72 aa, Phyllomedusa bicolor
  MOD_RES (72)..(72): OPTIONAL AMIDATION
MAFLKKSLFLVLFLGLVSLSVCEEEKRENEDEMEQEDDEQSEEKRALWKDILKNAGKAALNEINQLVNQGEL

SEQ ID NO: 27 - protein, 74 aa, Phyllomedusa bicolor
  MOD_RES (74)..(74): OPTIONAL AMIDATION
MAFLKKSVFLVLFLGLVSLSICEEEKREEENEEKQEDDEQSEEKRALWKNMLKGIGKLAGQAALGAVKTLVGAE

SEQ ID NO: 28 - protein, 77 aa, Phyllomedusa bicolor
  MOD_RES (77)..(77): OPTIONAL AMIDATION
MAFLKKSLFLVLFLGLVPLSLCESEKREGENEEEQEDDQSEEKRSLGSFLKGVGTTLASVGKVVSDQFGKLLQAGQG

SEQ ID NO: 29 - protein, 26 aa, Phyllomedusa bicolor
  MOD_RES (26)..(26): OPTIONAL AMIDATION
GLWNKIKEAASKAAGKAALGFVNEMV

SEQ ID NO: 30 - protein, 27 aa, Phyllomedusa bicolor
  MOD_RES (27)..(27): OPTIONAL AMIDATION
DVLKKIGTVALHAGKAALGAVADTISQ

SEQ ID NO: 31 - protein, 21 aa, Atractaspis microlepidota
CSCNDMNDKECMYFCHQDVIW

SEQ ID NO: 32 - protein, 21 aa, Artificial
  Synthesized sarafotoxin
CSCKDMSDKECLNFCHQDVIW

SEQ ID NO: 33 - protein, 33 aa, Phyllomedusa bicolor
  MOD_RES (33)..(33): OPTIONAL AMIDATION
GLWSKIKEVGKEAAKAAAKAAGKAALGAVSEAV

SEQ ID NO: 34 - protein, 33 aa, Artificial; identical to SEQ ID NO: 33
  Synthesized adenoregulin
  MOD_RES (33)..(33): OPTIONAL AMIDATION

SEQ ID NO: 35 - protein, 32 aa, Artificial
  Synthesized adenoregulin
  MOD_RES (32)..(32): OPTIONAL AMIDATION
GLWSKIKEVGKEAAKAAAKAAGKAALGAVSEA

SEQ ID NO: 36 - protein, 33 aa, Artificial
  Synthesized adenoregulin
  MOD_RES (33)..(33): OPTIONAL AMIDATION
GLWSKIKEVGKEAAKAAAKAAGKAALGAVSEAF

SEQ ID NO: 37 - protein, 33 aa, Artificial
  Synthesized adenoregulin
  MOD_RES (31)..(31): OPTIONAL AMIDATION
GLWSKIKEVGKEAAKAAAKAAGKAALGAVSEAL

SEQ ID NO: 38 - protein, 33 aa, Artificial
  Synthesized adenoregulin
  MOD_RES (33)..(33): OPTIONAL AMIDATION
GLWSKIKEVGKEAAKAAAKAAGKAALGAVSEAI

SEQ ID NO: 39 - protein, 33 aa, Artificial
  Synthesized adenoregulin
  MOD_RES (33)..(33): OPTIONAL AMIDATION
GLWSKIKEVGKEAAKAAAKAAGKAALGAVSEAY

SEQ ID NO: 40 - protein, 33 aa, Artificial
  Synthesized adenoregulin
  MOD_RES (33)..(33): OPTIONAL AMIDATION
GLWSKIKEVGKEAAKAAAKAAGKAALGAVSEAW

SEQ ID NO: 41 - protein, 33 aa, Artificial
  Synthesized adenoregulin
  MOD_RES (33)..(33): OPTIONAL AMIDATION
GLWSKIKEVGKEAAKAAAKAAGKAALGAVSEAA

SEQ ID NO: 42 - protein, 33 aa, Artificial
  Synthesized adenoregulin
  MOD_RES (33)..(33): OPTIONAL AMIDATION
GLWSKIKEVGKEAAKAAAKAAGKAALGAVSEAK

SEQ ID NO: 43 - protein, 33 aa, Artificial
  Synthesized adenoregulin
  MOD_RES (33)..(33): OPTIONAL AMIDATION
GLWSKIKEVGKEAAKAAAKAAGKAALGAVSEAP

SEQ ID NO: 44 - protein, 33 aa, Artificial
  Synthesized adenoregulin
  MOD_RES (33)..(33): OPTIONAL AMIDATION
GLWSKIKEVGKEAAKAAAKAAGKAALGAVSEAR

SEQ ID NO: 45 - protein, 33 aa, Artificial
  Synthesized adenoregulin
  MOD_RES (33)..(33): OPTIONAL AMIDATION
GLWSKIKEVGKEAAKAAAKAAGKAALGAVSEAH

SEQ ID NO: 46 - protein, 32 aa, Artificial; identical to SEQ ID NO: 35
  Synthesized adenoregulin
  MOD_RES (32)..(32): OPTIONAL AMIDATION

SEQ ID NO: 47 - protein, 36 aa, Artificial
  Synthesized adenoregulin
  MOD_RES (36)..(36): OPTIONAL AMIDATION
GLWSKIKEVGKEAAKAAAKAAGKAALGAVSEAVGEQ

SEQ ID NO: 48 - protein, 33 aa, Artificial
  Synthesized adenoregulin
  MOD_RES (33)..(33): OPTIONAL AMIDATION
GLWSKIKEVGKEAAKAAAKAAIKAALIAVSEAV

SEQ ID NO: 49 - protein, 32 aa, Artificial; identical to SEQ ID NO: 35
  Synthesized adenoregulin
  MOD_RES (32)..(32): OPTIONAL AMIDATION

SEQ ID NO: 50 - protein, 33 aa, Artificial
  Synthesized adenoregulin
  MOD_RES (33)..(33): OPTIONAL AMIDATION
GLWSAIKEVGKEAAKAAAKAAGKAALGAVSEAV

SEQ ID NO: 51 - protein, 33 aa, Artificial
  Synthesized adenoregulin
  MOD_RES (33)..(33): OPTIONAL AMIDATION
GLASKIKEVGKEAAKAAAKAAGKAALGAVSEAV

SEQ ID NO: 52 - protein, 33 aa, Artificial
  Synthesized adenoregulin
  MOD_RES (33)..(33): OPTIONAL AMIDATION
ALWSKIKEVGKEAAKAAAKAAGKAALGAVSEAV

SEQ ID NO: 53 - protein, 33 aa, Artificial
  Synthesized adenoregulin
  MOD_RES (33)..(33): OPTIONAL AMIDATION
GLWSKIKEVGKEAAKAAAKAAGKAALGAASEAV

SEQ ID NO: 54 - protein, 33 aa, Artificial
  Synthesized adenoregulin
  MOD_RES (33)..(33): OPTIONAL AMIDATION
GAWSKIKEVGKEAAKAAAKAAGKAALGAVSEAV

SEQ ID NO: 55 - protein, 33 aa, Artificial; identical to SEQ ID NO: 41
  Synthesized adenoregulin
  MOD_RES (33)..(33): OPTIONAL AMIDATION

SEQ ID NO: 56 - protein, 33 aa, Artificial
  Synthesized adenoregulin
  MOD_RES (33)..(33): OPTIONAL AMIDATION
GLWAKIKEVGKEAAKAAAKAAGKAALGAVSEAV

SEQ ID NO: 57 - protein, 33 aa, Artificial
  Synthesized adenoregulin
  MOD_RES (33)..(33): OPTIONAL AMIDATION
GLWSKIKEVGKEAAKAAAKAAGKAALGAVAEAV

SEQ ID NO: 58 - protein, 33 aa, Artificial
  Synthesized adenoregulin
  MOD_RES (33)..(33): OPTIONAL AMIDATION
GLWSKIKEVGKEAAKAAAKAAGKAALGAVSAAV

SEQ ID NO: 59 - protein, 34 aa, Artificial
  Synthesized adenoregulin
  MOD_RES (34)..(34): OPTIONAL AMIDATION
ALWKTMLKKLGTMALHAGKAALGAAADTISQGTQ

SEQ ID NO: 60 - protein, 31 aa, Artificial
  Synthesized adenoregulin
  MOD_RES (31)..(31): AMIDATION
ALWKTLLKKVGKVAGKAVLNAVTNMANQNEQ

SEQ ID NO: 61 - protein, 27 aa, Artificial
  Synthesized adenoregulin
  MOD_RES (27)..(27): OPTIONAL AMIDATION
ALWMTLLKKVLKAAAKALNAVLVGANA

SEQ ID NO: 62 - protein, 13 aa, Artificial
  Synthesized adenoregulin
  MOD_RES (13)..(13): OPTIONAL AMIDATION
ALWKTLLKKVLKA

SEQ ID NO: 63 - protein, 36 aa, Artificial
  Synthesized adenoregulin
  MOD_RES (36)..(36): OPTIONAL AMIDATION
GMWSKIKNAGKAAAKASKKAAGKAALGAVSEALGEQ

SEQ ID NO: 64 - protein, 27 aa, Artificial
  Synthesized adenoregulin
  MOD_RES (27)..(27): OPTIONAL AMIDATION
GMWGSLLKGVATVVKHVLPHALSSQQS

(19) United States
(12) Patent Application Publication
(10) Pub. No.: US 2010/0004166 A1
(43) Pub. Date: Jan. 7, 2010

(54) Endothelin and Endothelin Receptor Agonists in the Treatment of Metabolic Diseases

(76) Inventors: Richard Pittner, San Diego, CA (US); Andrew Young, San Diego, CA (US)

Correspondence Address: Intellectual Property Department;Amylin Pharmaceuticals, Inc., 9360 Towne Centre Drive, San Diego, CA, 92121, US

(21) Appl. No.: 12/294,235

(22) PCT Filed: Mar. 23, 2007
(86) PCT No.: PCT/US07/07376

§ 371 Date: Feb. 17, 2009

(51) Int. Cl. A61K 38/17; A61P 3/00

(52) U.S. Cl. 514/12

(57) ABSTRACT

Methods for treating conditions or disorders which can be alleviated by reducing food intake are disclosed which comprise administration of an effective amount of an endothelin or an endothelin agonist, alone or in conjunction with other compounds or compositions that affect satiety. The methods are useful for treating conditions or disorders, including obesity, Type II diabetes, eating disorders, and insulin-resistance syndrome. Pharmaceutical compositions for use in the methods of the invention are also disclosed.

### CROSS REFERENCE TO RELATED APPLICATIONS

This application claims the benefit of U.S. Provisional Application Ser. No. 60/785,447, filed Mar. 23, 2006 and which is incorporated herein by reference in its entirety.

### TECHNICAL FIELD

The present invention relates to the field of medicine and health. More particularly, the present invention relates to methods and compositions for reducing food intake, weight loss or treating metabolic diseases such as diabetes and obesity in a subject using at least one endothelin or endothelin receptor agonist.

### BACKGROUND

Obesity affects millions of people, particularly in developed countries such as the United States. According to the World Health Organization, more than one billion adults worldwide are overweight and at least 300 million of those are obese (having a Body Mass Index (BMI) of greater than 30). Smyth et al., Nature Medicine 12:75-80 (2005). In the United States alone, approximately 65% of adults are overweight or obese. Alarmingly, overweight and obesity also affect an estimated 10% of children worldwide. Yach et al., Nature Medicine 12:62-66 (2005). Excessive weight predisposes individuals to a myriad of conditions and diseases that include cardiovascular disease, stroke, osteoarthritis, type 2 diabetes, sleep apnea, respiratory disease, gastroesphogeal reflux disease, metabolic syndrome, cancer, as well as psychological consequences such as low self-esteem and clinical depression. Importantly, obesity also significantly increases the likelihood of morbidity and mortality from virtually all other diseases.

While the pathogenesis of obesity is often multifactorial, the fundamental cause lies in a caloric (i.e., food or nutrient) intake that is disproportionately high relative to the energy expenditure. Excess calories result in increased fat or adipose tissue production. The sustained loss of such adipose tissue can reduce and even eliminate the dire consequences of obesity. Typically, restricting total caloric intake either alone or in combination with increasing energy expenditure through, for example, increased exercise, results in reduced adipose tissue.

Despite the simplicity of the solution, sustained weight loss in overweight and obese individuals remains extraordinarily difficult, particularly for the obese patient. Treatment strategies range from diet and exercise regimens, behavioral modification techniques, and pharmacotherapy such as appetite suppressants and food absorption inhibitors to gastric bypass surgery and mechanical devices such as jaw wiring, waist cords, and balloons. Ultimately, the goal of each of these strategies is a sustained reduction in food or nutrient intake. However, a variety of factors complicate attaining the goal of sustained, reduced food intake. Some treatments simply aren't feasible for the extremely obese. For example, these individuals are often too large to safely exercise or are poor candidates for surgical intervention. Also while the loss of body fat is desired, the loss of lean body mass is not. Lean body mass constitutes muscle, vital organs, bone, connective and other non-fatty tissues in the body, and its loss is deleterious to the health of an individual. Yet, many treatments result in a simultaneous loss of adipose tissue and lean body mass, leaving the individual weakened by the treatment. Additional factors complicating sustained reductions in food intake include increased appetite and decreased energy levels following significant caloric restrictions.

Endothelins are key players in the health and maintenance of the cardiovascular system. See, e.g., Miyauchi, Ann. Rev. Physiol. 61:391-415 (1999). One group suggested targeting the endothelin system for the treatment of metabolic disorders such as obesity and diabetes using methods and compositions that antagonize or block the expression of endothelins by interfering with the enzymatic activity of endothelin-converting enzyme-2 (ECE-2). See e.g. U.S. Patent Application No. 2003/0232044. In other words, treatment of metabolic disorders was achieved by reducing or eliminating endothelin activity using the modulators of ECE-2 activity.

With continually increasing numbers of obese individuals worldwide, more effective methods to reduce food intake and induce weight loss, as well as to maintain weight loss over the long-term, are in great need. Endothelins and endothelin receptor agonists offer a new therapeutic alternative for conditions and disorders benefited by the reduction of food intake.

### BRIEF SUMMARY

Provided herein are methods and compositions for treating conditions or disorders which can be alleviated by reducing food intake that comprise the administration of an effective amount of an endothelin or an endothelin agonist, alone or in conjunction with other compounds or compositions that reduce food intake, induce satiety or otherwise enhance weight loss or maintenance. Such methods are useful in treating conditions including obesity, type II diabetes, metabolic syndrome, and insulin-resistance syndrome.

Thus, in one aspect, provided herein are methods for reducing food intake in subjects desirous or in need thereof by administering an endothelin or endothelin agonist in an amount effective to reduce food intake.

In another aspect, provided herein is a method to reduce or to maintain body weight in a subject desirous or in need thereof comprising administering an endothelin or an endothelin agonist in an amount effective to reduce or to maintain body weight.

Further provided herein is a method to treat obesity in a subject desirous or in need thereof comprising administering an endothelin or an endothelin agonist in an amount effective to treat obesity.

Also provided herein is a method to prevent or treat a metabolic disorder in a subject desirous or in need thereof, comprising administering an endothelin or an endothelin agonist effective in an amount to reduce or alleviate at least one symptom of said metabolic disorder. In some embodiments, the metabolic disorder is obesity, diabetes mellitus, insulin-resistant syndrome, syndrome-X, or disorders associated with excess caloric intake.

In one embodiment of the above methods, the endothelin is at least one of an endothelin-1, for example SEQ ID NOs: 1-6; an endothelin-2, for example SEQ ID NOs: 7-9; or an endothelin-3, for example SEQ ID NOs: 10-13. In one embodiment, the endothelin is endothelin-3. In another embodiment, the endothelin is any one of SEQ ID NOs: 1, 7, 10 or any combination thereof. In another embodiment, the endothelin is any one of SEQ ID NOs: 1-13 or any combination thereof, including each subset which specifically excludes one or more of SEQ ID NOs 1-13. In yet another embodiment, the endothelin has an amino acid sequence at least, 75%, at least 80%, at least 85%, at least 90% or 95% identical to any one of SEQ ID NOs: 1, 7 or 10. In still another embodiment, the endothelin agonist is an endothelin analog containing not more than 5 amino acid substitutions, deletions or additions, or not more than 10 amino acid substitutions, deletions or additions, as compared to any one of SEQ ID NOs: 1, 7 or 10 In another embodiment, the endothelin agonist is an endothelin analog containing no more than 5 amino acid substitutions as compared to any one of SEQ ID NOs: 1, 7, or 10.

In other embodiments, the endothelin agonist is a fragment of an endothelin-1, endothelin-2 or endothelin-3, wherein the fragment binds to and activates an ET_{A} or ET_{B} receptor. In another embodiment, the fragment preferentially binds to and activates an ET_{B} receptor; while in one embodiment, the fragment binds to and activates an ET_{B} receptor, but does not activate an ET_{A} receptor. In one embodiment the fragment does not contain more than 7 amino acid deletions as compared to a full length endothelin, for example, SEQ ID NOs: 1-13.

In yet another aspect, the endothelin receptor agonist is a sarafotoxin, for example any one of SEQ ID NO: 15, 16, 17, 18, 31 or 32. Also included is any combination of SEQ ID NO: 15, 16, 17, 18, 31 or 32 including any subset of thereof which may specifically exclude one of more of the preceding SEQ ID NOs. In yet another embodiment, the sarafotoxin has at least 80%, at least 85%, at least 90% or 95% amino acid sequence identity to any one of SEQ ID NOs: 15, 16, 17, 18, 31 or 32. In another embodiment, the sarafotoxin contains not more that 10, or not more than 5, amino acid deletions, additions or substitutions as compared to any one of SEQ ID NOs: 15; 16, 17, 18, 31 or 32. In one embodiment, the sarafotoxin preferentially binds to and activates an ET_{B} receptor.

In a further embodiment, the endothelin agonist is a dermaseptin and in a more specific embodiment, the dermaseptin is an adenoregulin. In one embodiment, the dermaspetin or adenoregulin has an amino acid sequence of any one of SEQ ID NOs: 20, 21, 22, 23, 24, 25, 26, 27, 28, 29, 30, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64 or any combination thereof, including any subset which may specifically exclude one or more of SEQ ID NOs 20-30 and 33-64. In yet another embodiment, the dermaseptin or adenoregulin has at least 75%, at least 80%, at least 85%, at least 90% or 95% amino acid sequence identity to any one of SEQ ID NOs: 20-30 or 33-64. In another embodiment, the dermaseptin or adenoregulin contains not more that 10, or not more than 5, amino acid deletions, additions or substitutions as compared to any one of SEQ ID NOs: 20-30 or 33-64. In one embodiment, the dermaseptin or adenoregulin preferentially binds to and activates an ET_{B} receptor.

The methods disclosed herein also can further comprise administration of a compound, wherein said compound induces satiety, reduces food intake or otherwise enhances weight loss or maintenance. Such compounds can include but are not limited to an exendin, amylin, PYY, leptin, oxyntomodullin, neuromedin or a cholecystokinin (CCK), their agonist analogs or derivatives thereof. In a specific embodiment, the endothelin agonist is adenoregulin, IRL1620, sarafotoxin S6c or any combination thereof.

In some embodiments, the endothelin or endothelin agonist acts via the ET_{B} receptor. In a specific embodiment, the endothelin or endothelin agonist does not act through the ET_{A} receptor.

Further provided herein is the use of a formulation comprising at least one endothelin or endothelin agonist to treat conditions or disorders that can be alleviated by reducing food intake as disclosed herewith, in an amount effective to treat said conditions. Also provided is the use of at least one endothelin or endothelin agonist to manufacture a medicament to mediate the effects or treat the diseases or disorders as disclosed herewith.

### BRIEF DESCRIPTION OF THE DRAWINGS

FIG. 1 depicts a dose-dependent reduction in food intake with endothelin receptor B (ET_{B}) agonists at 120 minutes.

FIG. 2 shows a dose-dependent reduction in food intake with ET_{B} agonists at 30 minutes.

FIG. 3 depicts a dose-dependent reduction in food intake with ET_{B} agonists at 60 minutes.

FIG. 4 shows a reduction of body fat with adenoregulin administration in diet-induced obese (DIO) mice maintained on a high-fat diet relative to mice maintained on a high-fat diet receiving vehicle only treatment or mice fed a low-fat diet and receiving vehicle only treatment.

FIG. 5 shows a reduction of body weight in diet-induced obese (DIO) mice maintained on a high fat diet and receiving 300 nmol/kg/d adenoregulin for 28 days. HF saline: DIO mice maintained on high fat diet and receiving vehicle (i.e., saline) only treatment. LF saline: DIO mice maintained on a low fat diet and receiving vehicle only treatment. DIO mice that received peptide YY (PYY) treatment while maintained on a high fat diet served as positive controls.

FIG. 6 shows a reduction in body fat in DIO mice maintained on a high fat diet and receiving 20 nmol/kg/d endothelin 1 (ET-1) treatment for 28 days via a subcutaneous (s.c.) osmotic pump.

### DETAILED DESCRIPTION

Provided herein is the use of endothelins and endothelin receptor agonists for reducing food intake or reducing or maintaining weight in subject desirous or in need thereof. Endothelins exert potent activities in numerous normal physiological and pathphysiological states. See, e.g., Kedzierski et al., Ann. Rev. Pharmacol. Toxicol. 41:851-76 (2001). Endothelins act as potent paracrine vasoconstrictor peptides that maintain basal vascular tone and regulate vascular growth. These peptides regulate tone in lung airways and blood vessels, control kidney water and sodium secretion as well as acid-base balance, and neurotransmissions. Conversely, endothelins also participate in the development and pathology of hypertension, artherosclerosis, cardiac hypertrophy, congestive heart failure, pulmonary hypertension, and renal failure. Factors stimulating endothelin production and activation include thrombin, TGF-β, TNF-α, norepinephrine, and insulin. See, e.g., Miyauchi et al., Ann. Rev. Physiol. 61:391-415 (1999). To date, however, the only role for endothelins in obesity and diabetes relates to the control of vascular complications and hypertension observed in patients with these diseases. See, e.g., Wolpert et al., Metabolism 42:1027-30 (1993); Ferri et al., Exp. Clin. Endocrinol. Diabetes 105(S2):38-40 (1997).

Disclosed herein is the discovery that systemic administration of endothelins acts to reduce food intake, decrease body weight, or both. Therefore, methods to reduce weight using endothelin and endothelin receptor agonists represent new therapeutic approaches to the treatment of metabolic disorders such as obesity, type II diabetes, insulin resistance, syndrome X, metabolic syndrome and other disorders associated with undesirable or excessive caloric intake. Reducing food intake in such subjects may also be useful in decreasing plasma glucose levels, plasma lipid levels, and risk of cardiovascular disease in these subjects.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

The term “condition or disorder which can be treated, alleviated, or prevented by reducing food intake” refers to any condition or disorder in a subject that is either caused by, complicated by, or aggravated by a relatively high food intake, or that can be alleviated or avoided by reducing food intake and/or body weight. Such conditions or disorders include, but are not limited to obesity, diabetes, including type II diabetes, insulin-resistance syndrome, and syndrome X. Reducing food intake can also aid in compliance with a dietary plan to control, reduce, or maintain weight, and to reduce daily caloric intake.

The term “endothelin agonist” refers to any isolated, naturally occurring or synthetic compound that binds to an endothelin receptor and mimics the food reduction intake action of endothelin at that receptor to result in a reduction of food intake. Thus, for the purposes of this application, endothelin agonist and endothelin receptor agonist are used interchangeably. In some embodiments, the endothelin agonist will specifically mimic one or more actions of endothelin-1, endothelin-2, or endothelin-3. Biological functions include initiating one or more signaling components, e.g., PLC activation, phospholipase A₂ activation, phospholipase D activation, MAPK activation, proto-oncogene expression (e.g., c-fos, c-myc, c-jun) or Shc-Grb2 complex formation. Such activities can be determined in vitro or in vivo according to standard techniques and assays. See, e.g., Badr et al., J. Clin. Invest. 83:336-42 (1989); Takuwa et al., J. Clin. Invest. 85:653-58 (1990); Resnik et al., Eur. J. Biochem. 189:415-21 (1990); Cazaubon et al., J. Biol. Chem. 269:24805-09 (1994); Wang et al., Am. J. Physiol. 267:C1130-35 (1994); Simonson et al., J. Biol. Chem. 267:8643-49 (1992).

As used herein, the term “obesity” or “obese” typically refers to an individual having a body mass index (BMI) of 30 kg/m² or more. See National Institute of Health, Clinical Guidelines on the Identification, Evaluation, and Treatment of Overweight and Obesity in Adults (1998). The term “overweight” describes an individual having a body mass index (BMI) of 25 kg/m² or greater, but less than 30 kg/m² or an individual who has a desire to lose weight regardless of their BMI. BMI is a measure expressing the relationship (or ratio) of weight-to-height based on a mathematical formula in which a person's body weight in kilograms is divided by the square of his or her height in meters (i.e., wt/(ht)²).

The term “metabolic disorder” refers to disorders, diseases, and conditions that are caused or characterized by abnormal energy use or consumption within the body. Examples of metabolic disease include, but are not limited to obesity, type II diabetes, insulin resistance syndrome, metabolic syndrome, and syndrome X.

As employed herein, the term “subject” embraces human as well as domestic and farm animals, nonhuman primates, and zoo animals, sports animals, or pet animals, such as dogs, cats, horses, cows, etc. and other mammals. It will be understood by the skilled practitioner that the subject is one appropriate to the desirability or need for food intake reduction, weight reduction, or maintenance of weight loss. In a specific embodiment, the subject is human.

The terms “treating” or “treatment” refer to the administration of one or more therapeutic agents to a subject who has a condition or disorder or a predisposition toward a condition or disorder, with the purpose to alleviate, relieve, alter, remedy, ameliorate, improve, affect, slow or stop the progression, slow or stop the worsening of the disease, at least one symptom of condition or disorder, or the predisposition toward the condition or disorder.

The endothelin system comprises three endothelins, two endothelin receptors, and two endothelin specific proteases. Endothelins exist in one of three isoforms: endothelin-1 (ET-1), endothelin-2 (ET-2), or endothelin-3 (ET-3). Each endothelin begins as a preproendothelin of about 200 amino acid residues. Furin-like endopeptidases cleave the preproendothelin at dibasic sites to form biologically inactive intermediates of about 37-41 amino acids known as big endothelins (big ETs) or pro-endothelins. Endothelin-converting enzymes (e.g., ECE-1 and ECE-2) then cleave the big ETs to form the 21 amino acid biologically active final product. Mammals express two endothelin receptors, ET_{A} and ET_{B}. These G-coupled receptors have seven transmembrane domains within their ˜400 amino acid sequence and induce a signaling cascade that includes phospholipase C-β activation, increased intracellular Ca²⁺, and immediate early gene expression. The receptors show differential affinities for the three ETs. ET_{A} has subnanomolar affinities for ET-1 and ET-2 (20-60 pm) but not ET-3 (6500 pm). ET_{B} has equipotent affinity for ET-1, ET-2 and ET-3. See, e.g., Arai et al., Nature 348:730-32 (1990); Sakurai et al., Nature 348:732-35 (1990); Sakurai et al., TiPS 13:103-08 (1992); U.S. Pat. No. 6,821,743.

ET-3/ET_{B} system appears functionally distinct from the ET-1/ET_{A} system. ET-1/ET_{A} system appears to mediate the proliferative and vasoconstrictive effects with ET_{A} expression observed throughout the vasculature. Thus, ET_{A} antagonists are frequently employed to restore hemostasis. ET_{B} expression, on the other hand, appears more restricted with expression in the gut, pituitary, brain, submandibular gland, brain, heart, kidney, jejunum, and stomach. See Matsumoto et al., Biochem. Biophys. Res. Commun. 164:74-80 (1989); Shiba et al., Biochem. Biophys. Res. Commun 186:588-94 (1992). ET-3/ET_{B} appears to primarily mediate vasorelaxation, but in some situations also mediates vasoconstriction and cellular proliferation.

Typically, circulating levels of endothelins are lower than that required for biological activity. For example, local ET-1 concentration within the vascular wall is ≧100-fold that of plasma levels. See, e.g., Kedzierski et al., Ann. Rev. Pharmacol. Toxicol. 41:851-76 (2001). The apparent requirement for high local concentrations for endothelin's vasoactive activities permits systemic administration of endothelin antagonists and agonists at lower doses to take advantage of other effects of the endothelins. In one instance, endothelin agonists successfully increased chemotherapy-mediated tumor regression in an animal following systemic administration without negative cardiovascular effects. See U.S. Patent Appl. No. 2004/0138121; Rajeshkumar et al., Breast Cancer Res. 94:237-47 (2005).

Any suitable source of ET-1, ET-2, or ET-3 may be used for the ET of the present methods. ETs may be isolated or purified from naturally-occurring sources, produced using molecular biology techniques (e.g., exogenous production), or using chemical synthesis. In one embodiment, ETs useful in the disclosed compositions and methods do not include big ETs (pro-ETs) or prepro-ETs. In another embodiment, ETs useful in the disclosed methods and compositions have an amino acid sequence containing 36 amino acids or less. Exemplary endothelins for use in the present methods and compositions are presented in Table 1. Typically endothelins contain 4 cysteine residues that form 2 sets of disulfide bonds between the two outer and two inner pairs of cysteines. For example, in SEQ ID NO: 1 disulfide bonds may be present between ¹C and ¹⁵C and between ³C and ¹¹C. In one embodiment, endothelins and endothelin agonists can be provided to the subject in an inactive (pro-drug) form comprising an endothelin, sarafotoxin, dermaseptin or adenoregulin described herein. Suitable pro-drug forms include a preproendothelin or a big ET.

TABLE 1
Exemplary Endothelins

| | | |
|---|---|---|
| Endothelin- | c (CSCSSLMDKECVYFCHLDIIW) | SEQ ID NO: 1 |
| 1 | c (CSCSSLLDKECVYFCHLDIIW) | SEQ ID NO: 2 |
| | c (CSCATFLDKECVYFCHLDIIW) | SEQ ID NO: 3 |
| | c (CSCASFLDKECVYFCHLDIIW) | SEQ ID NO: 4 |
| | c (CSCSSLLDEECVYFCHLDIIW) | SEQ ID NO: 5 |
| | c (CSCSSLLDKECVYFCLLDIIW) | SEQ ID NO: 6 |
| Endothelin- | c (CSCSSWLDKECVYFCHLDIIW) | SEQ ID NO: 7 |
| 2 | c (CSCNSWLDKECVYFCHLDIIW) | SEQ ID NO: 8 |
| | c (CSCNSWLDKECIYFCHLDIIW) | SEQ ID NO: 9 |
| Endothelin- | c (CTCFTYKDKECVYYCHLDIIW) | SEQ ID NO: 10 |
| 3 | c (CTCYTYKDKECVYYCHLDIIW) | SEQ ID NO: 11 |
| | c (CTCFTYKDRECVYYCHLDIIW) | SEQ ID NO: 12 |
| | c (CTCYSYKDKECVYYCHLDIIW) | SEQ ID NO: 13 |

Exemplary ET_{B} agonists include but are not limited to analogs, derivatives, or functional fragments of ET-1, ET-2, or ET-3. Further, representative sequences for the endothelins include, but are not limited to those disclosed in Inoue et al., Proc. Natl. Acad. Sci. USA 86:2863-67 (1989); and U.S. Pat. Nos. 5,294,569; 5,231,166; and 4,981,950, which are incorporated herein by reference. The analog, derivative or fragment can be made using standard molecular biology techniques or chemical synthesis, and is one that retains the biological function or activity, e.g., reducing food intake, reducing body weight, or both as described herein. In one embodiment, the agonist binds ET_{B} receptor. In a specific embodiment, the agonist selectively binds the ET_{B} receptor and does not bind the ET_{A} receptor sufficiently to elicit detectable ET_{A} activation in standard assays. In one embodiment, the agonist does not bind the ET_{A} receptor. In some embodiments, the endothelin agonist binds the ET_{B} receptor with a greater affinity than the ET_{A} receptor, for example with at least 5-fold, 10-fold, 25-fold, 100-fold, 200-fold or 1000-fold greater affinity.

ET_{B} agonists useful in the disclosed methods include, but are not limited to BQ-788 (Clinalfa AG; N-cis-2,6-dimethylpiperidinocarbonyl-L-γ-methylleucyl-D-1-methoxycarbonyltryptophanyl-D-norleucine; Ishikawa et al., Proc. Natl. Acad. Sci. USA 91:4892 (1994)), IRL1620 (N-Succinyl-[Glu⁹, Ala^{11,15}]-Endothelin 1 fragment 8-21; Takai, et al., Biochem. Biophys. Res. Commun. 184:953 (1992)), BQ-3020 (N-Acetyl-[Ala^{11,15}]-Endothelin 1 fragment 6-21; Ihara, et al., Life Sci. 51:PL47-PL52, (1992)), and [Ala^{1,3,11,15}]ET-1 (ASASSLMDKEAVYFAHLDIIW [SEQ ID NO: 14]).

Another source of ET_{B} agonists are the sarafotoxins. Isolated from a burrowing asp, Atractaspic engadensisan, this pro-coagulant toxin binds preferentially with ET_{B}. See Takasaki et al., Toxicon 26:543-48 (1988). Some exemplary sarafotoxin ET_{B} agonists are disclosed below in Table 2. Additional sarafotoxins include CSCNDMNDKECMYFCHQDVIW (SEQ ID NO: 31) and CSCKDMSDKECLNFCHQDVIW (SEQ ID NO: 32). Like endothelins, disulfide bonds may be present between the outer and inner pairs of sarafotoxin cysteine residues.

TABLE 2
Exemplary Sarafotoxin ET_{B} Agonists

| | |
|---|---|
| Sarafotoxin S6a | c (Cys-Ser-Cys-Lys-Asp-Met-Thr- |
| | Asp-Lys-Glu-Cys-Leu-Asn-Phe-Cys- |
| | His-Gln-Asp-Val-Ile-Trp) |
| | [SEQ ID NO: 15] |
| Sarafotoxin S6b | c (Cys-Ser-Cys-Lys-Asp-Met-Thr- |
| | Asp-Lys-Glu-Cys-Leu-Tyr-Phe-Cys- |
| | His-Gln-Asp-Val-Ile-Trp) |
| | [SEQ ID NO: 16] |
| Sarafotoxin S6c | c (Cys-Thr-Cys-Asn-Asp-Met-Thr- |
| | Asp-Glu-Glu-Cys-Leu-Asn-Phe-Cys- |
| | His-Gln-Asp-Val-Ile-Trp) |
| | [SEQ ID NO: 17] |
| Sarafotoxin S6d | c (Cys-Thr-Cys-Asn-Asp-Met-Thr- |
| | Asp-Lys-Glu-Cys-Leu-Tyr-Phe-Cys- |
| | His-Glu-Asp-Ile-Ile-Trp |
| | [SEQ ID NO: 18] |

In yet another embodiment, the ET receptor agonist is adenoregulin. See, e.g., Donly et al., Proc. Natl. Acad. Sci. USA 89:10960-963 (1992); Amiche et al., Biochem. Biophys. Res. Comm. 191:983-90 (1993). Known sequences for adenoregulin include Genbank sequences X70278 and X72387. Originally isolated from the skin of the frog Phyllomedusa bicolor, adenoregulin belongs to the family of dermaseptins, a family of antimicrobial peptides. See, e.g., Amiche et al., J. Biol. Chem. 269:17847-52 (1994); Zhou et al., Biotechnol. Lett. 27:725-30 (2005). Exemplary adenoregulins are presented in Table 3. Examples of reduction in food intake by administration of additional exemplary adenoregulins is presented in Table 4. In one embodiment, the endothelin receptor agonist is one exhibiting at least a 15%, at least a 25%, at least a 30%, at least a 40%, at least a 50%, at least a 60% or at least a 70% reduction in food intake at 120 minutes post administration as compared to vehicle or no treatment.

TABLE 3
Exemplary Adenoregulins

| | |
|---|---|
| SEQ ID NO: 20 | MAFLKKSLFLVLFLGLVSLSICEEEKRENEDE |
| | EEQEDDEQSEMKRGLWSKIKEVGKEAAKAAAK |
| | AAGKAALGAVSEAVGEQ |
| SEQ ID NO: 21 | MDILKKSLFLVLFLGLVSLSICEEEKRENEDE |
| | EKQDDEQSEMKRAMWKDVLKKIGTVALHAGKA |
| | ALGAVADTISQGEQ |
| SEQ ID NO: 22 | MAFLKKSLFLVLFLGLVSLSICEEEKRENEDE |
| | EEQEDDEQSEMKRGLWSKIKEAGKAALTAAG |
| | KAALGAVSDAVGEQ |
| SEQ ID NO: 23 | MAFLKKSLFLVLFLGLVSLSICEEEKRENKDE |
| | IEQEDDEQSEEKRALWKDILKNVGKAAGKAVL |
| | NTVTDMVNQGEQ |
| SEQ ID NO: 24 | MASLKKSLFLVLFLGLVSLSICEEEKRENED |
| | EEEQEDDEQSEMKRGLWSNIKTAGKEAAKAAL |
| | KAAGKAALGAVTDAVGEQ |
| SEQ ID NO: 25 | MDILKKSLFLVLFLGLVSLSICEEEKRENEDE |
| | EKQDDEQSEMKRAMWKDVLKKIAGKAALGAVA |
| | DTISQGEQ |
| SEQ ID NO: 26 | MAFLKKSLFLVLFLGLVSLSVCEEEKRENEDE |
| | MEQEDDEQSEEKRALWKDILKNAGKAALNEIN |
| | QLVNQGEL |
| SEQ ID NO: 27 | MAFLKKSVFLVLFLGLVSLSICEEEKREEENE |
| | EKQEDDEQSEEKRALWKNMLKGIGKLAGQAAL |
| | GAVKTLVGAE |
| SEQ ID NO: 28 | MAFLKKSLFLVLFLGLVPLSLCESEKREGENE |
| | EEQEDDQSEEKRSLGSFLKGVGTTLASVGKVV |
| | SDQFGKLLQAGQG |
| SEQ ID NO: 29 | GLWNKIKEAASKAAGKAALGFVNEMV |
| SEQ ID NO: 30 | DVLKKIGTVALHAGKAALGAVADTISQ |

TABLE 4

Percent Inhibition of Food Intake (FI) at
120 min by Adenoregulins

| | | |
|---|---|---|
| SEQ | | % Inhibition of |
| ID NO | Sequence | FI at 120 mi |

| | | |
|---|---|---|
| 33 | GLWSKIKEVGKEAAKAAAKAAGKAALGAVSEAV-NH2 | 77.6 |
| 34 | GLWSKIKEVGKEAARAAAKAAGKAALGAVS-NH2 | 49.6 |
| 35 | GLWSKIKEVGKEAAKAAAKAAGKAALGAVSEA-NH2 | 28.9 |
| 36 | GLWSKIKEVGKEAAKAAAKAAGKAALGAVSEAF-NH2 | 92.0 |
| 37 | GLWSKIKEVGKEAAKAAAKAAGKAALGAVSEAL-NH2 | 79.0 |
| 38 | GLWSKIKEVGKEAAKAAAKAAGKAALGAVSEAI-NH2 | 78.0 |
| 39 | GLWSKIKEVGKEAAKAAAKAAGKAALGAVSEAY-NH2 | 61.0 |
| 40 | GLWSKIKEVGKEAAKAAAKAAGKAALGAVSEAW-NH2 | 61.0 |
| 41 | GLWSKIKEVGKEAAKAAAKAAGKAALGAVSEAA-NH2 | 54.0 |
| 42 | GLWSKIKEVGKEAAKAAAKAAGKAALGAVSEAK-NH2 | 48.0 |
| 43 | GLWSKIKEVGKEAAKAAAKAAGKAALGAVSEAP-NH2 | 37.0 |
| 44 | GLWSKIKEVGKEAAKAAAKAAGKAALGAVSEAR-NH2 | 34.0 |
| 45 | GLWSKIKEVGKEAAKAAAKAAGKAALGAVSEAH-NH2 | 23.0 |
| 46 | GLWSKIKEVGKEAAKAAAKAAGKAALGAVSEA-NH2 | 17.0 |
| 47 | GLWSKIKEVGKEAAKAAAKAAGKAALGAVSEAVGEQ-OH | 50.0 |
| 48 | GLWSKIKEVGKEAAKAAAKAAIKAALIAVSEAVV-NH2 | 47.9 |
| 49 | GLWSKIKEVGKEAAKAAAKAAGKAALGAVSEA-NH2 | 13.5 |
| 50 | GLWSAIKEVGKEAAKAAAKAAGKAALGAVSEAV-NH2 | 22.3 |
| 51 | GLASKIKEVGKEAAKAAAKAAGKAALGAVSEAV-NH2 | 25.8 |
| 52 | ALWSKIKEVGKEAAKAAAKAAGKAALGAVSEAV-NH2 | 32.3 |
| 53 | GLWSKIKEVGKEAAKAAAKAAGKAALGAASEAV-NH2 | 33.6 |
| 54 | GAWSKIKEVGKEAAKAAAKAAGKAALGAVSEAV-NH2 | 37.3 |
| 55 | GLWSKIKEVGKEAAKAAAKAAGKAALGAVSEAA-NH2 | 54.0 |
| 56 | GLWAKIKEVGKEAAKAAAKAAGKAALGAVSEAV-NH2 | 71.7 |
| 57 | GLWSKIKEVGKEAAKAAAKAAGKAALGAVAEAV-NH2 | 74.8 |
| 58 | GLWSKIKEVGKEAAKAAAKAAGKAALGAVSAAV-NH2 | 78.9 |
| 59 | ALWKTMLKKLGTMALHAGKAALGAAADTISQGTQ-OH | 58.0 |
| 60 | ALWKTLLKKVGKVAGKAVLNAVTNMANQNEQ-OH | 53.0 |
| 61 | ALWMTLLKKVLKAAAKALNAVLVGANA-OH | 35.0 |
| 62 | ALWKTLLKKVLKA-NH2 | 28.2 |
| 63 | GMWSKIKNAGKAAAKASKKAAGKAALGAVSEALGEQ-OH | 50.0 |
| 64 | GMWGSLLKGVATVVKHVLPHALSSQQS-OH | 22.0 |

Additional ET receptor agonists are readily identifiable using methods known in the art. See, e.g., Davenport, Pharmacological Rev. 54:219-26 (2002); U.S. Pat. No. 6,821,743. For example, an agonist can readily be identified using in vitro or in vivo analysis of known endothelin induced activities in cells expressing ET_{A}, ET_{B}, or both. Such cells can express the receptors either endogenously or exogenously using known molecular biology techniques. In one embodiment, a selective ET_{B} agonist would not bind or cause measurable activation of ET_{A}. Conversely, in another embodiment, a selective ET_{A} agonist would not bind or cause measurable activation of ET_{B}.

Such agonists can encompass numerous chemical classes. In certain embodiments, they are organic molecules, preferably small organic compounds having a molecular weight of more than 50 and less than about 2,500 daltons. ET receptor agonists also include biomolecules like antibodies, peptides, polypeptides, peptidomimetics, saccharides, fatty acids, steroids, purines, pyrimidines, derivatives, structural analogs or combinations thereof. In some embodiments, ET receptor agonists can include peptide and protein agents, such as antibodies or binding fragments or mimetics thereof, e.g., Fv, F(ab′)₂ and Fab. Known pharmacological agents may be subjected to directed or random chemical modifications, such as acylation, alkylation, esterification, amidification, etc. to produce structural analogs that act as non-selective or selective ET receptor agonists.

In some embodiments, ET receptor agonists are endothelin variants such as analogs or functional fragments of ET-1, ET-2, or ET-3. A functional fragment is an endothelin having less than 21 amino acids, but which still binds to and activates an endothelin receptor resulting in a decrease in food intake, and/or body weight. Analogs include those having five or less amino acid substitutions as well as those with at least 75%, 80%, 85%, 90%, or 95% amino acid sequence identity with the sequence of SEQ ID NO: 1, SEQ ID NO: 7 or SEQ ID NO: 10. In another embodiment, the endothelin variant has not more than 10, not more than 7, not more than 5, not more than 4, not more than 3, not more than 2 or only a single amino acid deletion, addition or substitution as compared to a known endothelin such as SEQ ID NO: 1, SEQ ID NO: 7 or SEQ ID NO: 10. Also contemplated are ET-1, ET-2, and ET-3 sequences from species other than human.

In some embodiments, the ET receptor agonists are variants such as analogs or functional fragments of a sarafotoxin or adenoregulin of Table 2 or 3 herein. A functional fragment can have less than 21 amino acids, but still bind to and activate an endothelin receptor resulting in a decrease in food intake, and/or body weight. Analogs include those having five or less amino acid substitutions as well as those with at least 75%, 80%, 85%, 90%, or 95% amino acid sequence identity with a sequence in Table 2 or 3. In another embodiment, the variant has not more than 10, not more than 7, not more than 5, not more than 4, not more than 3, not more than 2 or only a single amino acid deletion, addition or substitution as compared to a sequence in Table 2 or 3. Also contemplated are their variant sequences from species other than human.

“Sequence Identity”, as is well understood in the art, is a relationship between two or more polypeptide sequences or two or more polynucleotide sequences, as determined by comparing the sequences. In the art, “identity” can also mean the degree of sequence relatedness between polypeptide or polynucleotide sequences, as determined by the match between strings of such sequences. Identity can be readily calculated by known methods including, but not limited to, those described in Computational Molecular Biology, Lesk, A. M., ed., Oxford University Press, New York (1988); Biocomputing: Informatics and Genome Projects, Smith, D. W., ed., Academic Press, New York, 1993; Computer Analysis of Sequence Data, Part I, Griffin, A. M. and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., Stockton Press, New York (1991); and Carillo, H., and Lipman, D., SIAM J Applied Math, 48:1073 (1988). Methods to determine identity are designed to give the largest match between the sequences tested. Moreover, methods to determine identity are codified in publicly available programs. Computer programs which can be used to determine identity between two sequences include, but are not limited to, GCG (Devereux, J., et al., Nucleic Acids Research 12(1):387 (1984); suite of five BLAST programs, three designed for nucleotide sequences queries (BLASTN, BLASTX, and TBLASTX) and two designed for protein sequence queries (BLASTP and TBLASTN) (Coulson, Trends in Biotechnology, 12: 76-80 (1994); Birren, et al., Genome Analysis, 1: 543-559 (1997)). The BLAST X program is publicly available from NCBI and other sources (BLAST Manual, Altschul, S., et al., NCBI NLM NIH, Bethesda, Md. 20894; Altschul, S., et al., J. Mol. Biol., 215:403-410 (1990)). The well known Smith Waterman algorithm can also be used to determine identity.

Parameters for polypeptide sequence comparison typically include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA 89:10915-10919 (1992); Gap Penalty: 12; Gap Length Penalty: 4. A program that can be used with these parameters is publicly available as the “gap” program from Genetics Computer Group (“GCG”), Madison, Wis. The above parameters along with no penalty for end gap are the default parameters for peptide comparisons. In one embodiment the BLASTP program of NCBI is used with the default parameters of no compositional adjustment, expect value of 10, word size of 3, BLOSUM62 matrix, gap extension cost of 11, end gap extension cost of 1, dropoff (X) for blast extension (in bits) 7, X dropoff value for gapped alignment (in bits) 15, and final X dropoff value for gapped alignment (in bits) 25.

Provided herein are methods for reducing food intake or reducing (or maintaining) body weight, or both in subjects desirous or in need thereof by administering at least one endothelin or agonist thereof. Such subjects are typically those with a condition or disorder or at risk of developing a condition or disorder that will benefit from the reduction of food intake, a reduction (or maintenance) in body weight, or both. Thus, in one aspect provided herein is a method to reduce body weight in a subject desirous or in need thereof comprising administering an effective amount of endothelin or an endothelin agonist. Further provided herein is a method to treat obesity in a subject in need thereof, comprising administering an effective amount of endothelin or an endothelin agonist. Also provided herein is a method to prevent or treat a metabolic disorder in a subject in need thereof, comprising administering an effective amount of endothelin or an endothelin receptor agonist to a subject with a metabolic disorder or at risk of having a metabolic disorder. In some embodiments, the metabolic disorder is obesity, diabetes mellitus, insulin-resistant syndrome, metabolic syndrome, syndrome-X, or other disorders resulting from excessive caloric intake or dysregulated food or energy metabolism. In one embodiment the subject having the metabolic disorder is also obese, overweight, desirous of reducing or maintaining body weight or desirous of preventing a further increase in body weight.

The endothelin can be an endothelin-1, an endothelin-2, an endothelin-3, an analog, derivative, or functional fragment thereof or combinations thereof. In a specific embodiment, the endothelin is human endothelin-3 (SEQ ID NO: 10). In some embodiments, the endothelin or endothelin agonist acts via the ET_{B} receptor. In a specific embodiment, the endothelin or endothelin agonist does not act through the ET_{A} receptor or has a reduced affinity for the ET_{A} receptor. In another embodiment, the endothelin or endothelin agonist acts preferentially through the ET_{B} receptor. In one embodiment, the subjects desirous or in need of treatment of conditions or disorders which can be alleviated by reducing food intake comprising administering an endothelin or an endothelin agonist in an amount effective to reduce food intake do not include subjects receiving an endothelin or an endothelin agonist for a cardiovascular indication.

Provided herein is a method to suppress appetite or induce satiety comprising administering an endothelin or an endothelin agonist to a subject desirous or in need thereof, in an amount effective to suppress appetite or induce satiety.

The methods disclosed herein also can further comprise administration of an additional compound, wherein said additional compound induces satiety, reduces food intake, or otherwise acts to maintain or reduce body weight. Such additional compounds can include but are not limited to an exendin or agonist thereof (see, e.g. U.S. Pat. No. 6,956,026; WO 99/25727; WO99/25728; WO99/07404); an amylin or agonist thereof (see, e.g., Pittner et al., J. Cell. Biochem. 55S:19-28 (1994); U.S. Pat. Nos. 5,686,411; 6,610,824; 6,410,511;); cholecystokinin (CCK) (see, e.g., U.S. Pat. Nos. 5,739,106; 5,270,302), oxyntomodulin (see, e.g., U.S. Pat. No. 5,858,975), peptide YY (PYY) (U.S. Patent Appl. Nos. 2005/0176643; 2002/0141985) or a leptin (ob protein) (see, e.g., U.S. Pat. Nos. 6,475,984; 6,399,745). Suitable amylin agonists include, e.g., [^{25,28,29} PRO-]-human amylin (also known as “pramlintide,”) (see, e.g., U.S. Pat. Nos. 5,175,145; 5,814,600; 5,998,367; 6,114,304; 6,410,511; 6,608,029; 6,6610,824), salmon calcitonin, and compounds described in US20050197287, WO2006105345, WO2006083254, WO2006052608, and WO2005115437, all of which are incorporated by reference for the amylin family compounds disclosed therein. The CCK used in one embodiment is CCK octopeptide (CCK-8). The endothelins and endothelin agonists may be administered separately or together with one or more of such compounds. The administration may be simultaneous, sequential, or serial in the same or a different composition.

Also included is the use of an endothelin or endothelin receptor agonist to reduce food intake, reduce (or maintain) body weight, or both in a subject desirous or in need thereof, comprising administering at least one endothelin or endothelin receptor agonist. Further provided is the use of an endothelin or endothelin receptor agonist to manufacture a medicament suitable for reducing food intake, reducing body weight, or both in a subject desirous or in need thereof.

In one aspect, the present methods relate to reducing body weight and/or reducing weight gain in an animal, and more particularly, to treating or ameliorating obesity in patients at risk for or suffering from obesity. In one embodiment, the method is effective to measurably decrease body weight in the animal. In some embodiments, a decrease in body weight in the animal can be measured within at least about two weeks of the step of administering the compound, sometimes at least about four weeks, but typically within at least 6-8 weeks.

In further embodiments, any of the methods disclosed herein result in the subject's body weight being reduced by at least 1%, at least 5%, at least 10%, at least 20%, at least 30%, at least 40% or at least 50%. In additional embodiments, any of the methods disclosed herein result in the subject's body weight being reduced by at least about 5 pounds or 2 kg, at least about 10 pounds or 5 kg, at least about 20 pounds or 10 kg, at least about 30 pounds or 15 kg, at least about 40 pounds or 20 kg, at least about 50 pounds or 25 kg, at least about 75 pounds or 35 kg, at least about 100 pounds or 50 kg, at least about 125 pounds or 55 kg, at least about 150 pounds or 75 kg, at least about 175 pounds or 80 kg, or at least about 200 pounds or 100 kg. In still further embodiments, practice of any of the methods disclosed herein results in weight reduction, wherein less than about 40%, less than about 20%, less than about 10%, less than about 5%, less than about 2%, less than about 1%, or 0% of the weight loss is due to loss of mean body mass.

In other embodiments, the subject's average food intake is reduced by at least 50% or the subject's average food intake is reduced by 40%, 35%, 30%, 25%, 20%, 15%, 10%, or 5% as compared to the subject's average food intake without administration of an endothelin or endothelin receptor agonists as described herein. Percent average food intake can be determined for any defined period, for example, on a per meal, daily, weekly or monthly basis. In another embodiment, the subject's average daily food intake is reduced by at least 2000 Calories, at least 1500 Calories or at least 1000 Calories as compared to the subject's food intake without administration of an endothelin or endothelin receptor agonists as described herein. In yet another embodiment, the subject's average daily food intake is reduced by 2000 Calories, 1500 Calories 1000, Calories, 750 Calories, 500 Calories, 250 Calories or 100 Calories as compared to the subject's food intake without administration of an endothelin or endothelin receptor agonists as described herein. As used herein, Calories refers to a nutritional calorie also known as a large calorie or kilogram calorie. A nutritional calorie is a unit expressing a heat-producing or energy-producing value in food that when oxidized in the body is capable of releasing one large calorie of energy (1000 gram calories or 3.968 Btu).

Conditions or disorders that can be treated or prevented in a patient by administering to the patient, an effective amount of compound of the present invention in such an amount and for such time as is necessary to achieve the desired result. The amount of an endothelin or endothelin agonist to effectively ameliorate disorders by reducing food intake or reducing (or maintaining) body weight is one at a reasonable benefit/risk ratio applicable to any medical treatment. Therefore, the dose would not be one that elicits undesirable cardiovascular effects for a patient. The specific effective dose level for any particular patient will depend upon a variety of factors including the condition or disorder being treated and the severity of the condition or disorder; the activity of the compound employed; the specific composition employed; the age, body weight, general health, sex, and diet of the patient; the time of administration, route of administration, rate of excretion; the duration of the treatment; and other active agents used in combination or coincidental therapy. For use by the physician, the compositions will be provided in dosage unit form containing an amount of an endothelin or endothelin agonist, with or without another food intake-reducing, plasma glucose-lowering or plasma lipid-lowering agent.

The term “an effective amount” refers to the amount of a compound alone or in combination according the methods disclosed herein required to reduce food intake, reduce body (or maintain) weight, or both. The effective daily food intake-reducing dose of the compounds will typically be in the range of about 10 μg to about 5 mg/day, about 10 μg to about 2 mg/day, about 10 μg to about 1 mg/day, or about 30 μg to about 500 μg/day, administered in a single or divided doses. In other embodiments the dose of an endothelin or sarafotoxin ranges from about 1 nmol/kg to about 10 micromol/kg, in another embodiment from about 1 nmol/kg to about 1000 mmol/kg, in another embodiment from about 10 nmol/kg to about 1000 nmol/kg, and in yet another embodiment from about 100 nmol/kg to about 1000 nmol/kg. In certain embodiments the dose of adenoregulins or dermaseptins will be 5-10 times greater than the doses for endothelins and sarafotoxins. Thus, in some embodiments, the dose of adenoregulin or sarafotoxin will range from about 5 nmol/kg to about 1 millimol/kg, in other embodiments from about 5 nmol/kg to about 10 micromol/kg, in other embodiments from about 50 mmol/kg to about 10 micromol/kg, in still other embodiments from about 500 nmol/kg to about 10 micromol/kg. In some embodiments, these doses are increased or decreased according to the body weight of the patient. Typically, administration of an endothelin or endothelin receptor agonist begins whenever the suppression of food intake, weight lowering, or weight maintenance is desired, for example, at the first sign of symptoms or shortly after diagnosis of obesity, diabetes mellitus, insulin-resistance syndrome or other conditions or disorders disclosed herein. Administration may be by injection, e.g., subcutaneous, intramuscular, intraperitoneal, or intravenous injection. Orally active compounds may be taken orally, however dosages are typically increased 5-10 fold. In some embodiments, the compound or composition is administered daily, weekly, or monthly. Dosages may be decreased as an individual's body weight decreases until a basal dosage is established or no dosage is required. In one embodiment the compound is administered immediately before a meal. In another embodiment, the compound is administered at the start of a meal.

In one aspect, an effective amount is the amount sufficient to cause a decrease in food intake, a decrease in body weight or both, without a significant or without a measurable effect on the cardiovascular system. Effects on the cardiovascular system include an increase or decrease in blood pressure, for example, systolic, diastolic or both; an increase in total peripheral vascular resistance; or an increase in pulmonary vascular resistance. In one embodiment, the amount is effective to cause a decrease in food intake, a decrease in body weight or both does not alter average systolic pressure, average diastolic pressure or both by more than 5 mm Hg. In another embodiment, the amount is effective to cause a decrease in food intake, a decrease in body weight or both does not increase average systemic systolic pressure, average systemic diastolic pressure or both by more than 5 mm Hg. In one embodiment, the amount is effective to cause a decrease in food intake, a decrease in body weight or both does not increase average systolic pressure to greater than 139 mm Hg or average diastolic pressure to greater than 89 mm Hg. In still another embodiment, the amount is effective to cause a decrease in food intake, a decrease in body weight or both does not increase the ratio of systolic to diastolic systemic blood pressure to greater than 139 over 89 mm Hg.

The data obtained from the cell culture assays and animal studies can be used in formulating a range of dosage for use in humans or other subject animals. The dosage of such compounds typically lies within a range of circulating concentrations that include the ED₅₀ with little or no toxicity. In particular, the present methods employ ET agonists that avoid harmful vascular effects, particularly in the cardiovascular system. The dosage may vary within this range depending upon the dosage form employed and the route of administration utilized. For any compound used in the method disclosed herein, the therapeutically effective dose can be estimated initially from cell culture assays. A dose may be formulated in animal models to achieve a circulating plasma concentration range that includes the EC₅₀ (i.e., the concentration of the test compound which achieves a half-maximal effective response) as determined in cell culture. Such information can be used to more accurately determine useful doses in humans. Levels in plasma may be measured by, for example, high performance liquid chromatography or antibody assays.

The optimal formulation and mode of administration of compounds of the present application to a patient depend on factors known in the art such as the particular condition or disorder, the desired effect, the type of patient, and patient preferences.

Compounds useful in the present methods may conveniently be provided in the form of formulations suitable for parenteral (including intravenous, intraperitoneal, intramuscular and subcutaneous) or nasal or oral administration. In one embodiment, the route of administration is subcutaneous or intramuscular. In some cases, it will be convenient to provide an endothelin or endothelin receptor agonist and at least one food-intake-reducing, plasma glucose-lowering or plasma lipid-lowering agent, such as exendin-4, an exendin agonist, amylin, an amylin agonist, PYY, a CCK, or a leptin, in a single composition or solution for administration together. In other cases, it may be more advantageous to administer the at least one additional agent separately from the endothelin or endothelin agonist. A suitable administration format may best be determined by a medical practitioner for each patient individually. Suitable pharmaceutically acceptable carriers and their formulation are described in standard formulation treatises. See, e.g. REMINGTON: THE SCIENCE AND PRACTICE OF PHARMACY (21st Ed. Lipincott, Williams, & Wilkins 2005).

As described herein, the endothelins and endothelin agonists may also be combined with any traditional method or compositions suitable to reduce food intake or to promote or maintain weight loss. Such methods and means include, but are not limited to diet, exercise, orlistat, sibutramine, bariatric surgery, and homeopathic appetite suppressing supplements.

Therapeutic compositions of the present compounds comprise an effective amount of the compound(s) formulated with one or more therapeutically suitable excipients. The term “therapeutically suitable excipient,” as used herein, represents a non-toxic, solid, semi-solid or liquid filler, diluent, encapsulating material, or formulation auxiliary of any type. Examples of therapeutically suitable excipients include sugars; cellulose and derivatives thereof; oils; glycols; solutions; buffering, coloring, releasing, coating, sweetening, flavoring, and perfuming agents; and the like. These therapeutic compositions can be administered parenterally, intracistemally, orally, rectally, or intraperitoneally.

Liquid dosage forms for oral administration of the present compounds comprise formulations of the same as emulsions, microemulsions, solutions, suspensions, syrups, and elixirs. In addition to the compounds, the liquid dosage forms can contain diluents and/or solubilizing or emulsifying agents. Besides inert diluents, the oral compositions can include wetting, emulsifying, sweetening, flavoring, and perfuming agents. Injectable preparations of the present compounds comprise sterile, injectable, aqueous and oleaginous solutions, suspensions or emulsions, any of which can be optionally formulated with parenterally suitable diluents, dispersing, wetting, or suspending agents. These injectable preparations can be sterilized by filtration through a bacterial-retaining filter or formulated with sterilizing agents that dissolve or disperse in the injectable media.

Compounds useful in the present methods can be provided as parenteral compositions for injection or infusion. They can, for example, be suspended in an inert oil, suitably a vegetable oil such as sesame, peanut, olive oil, or other acceptable carrier. These compositions may be sterilized by conventional sterilization techniques, or may be sterile filtered. The compositions may contain pharmaceutically acceptable auxiliary substances as required to approximate physiological conditions, such as pH buffering agents. Useful buffers include for example, sodium acetate/acetic acid buffers. A form of repository, sustained or “depot” slow release preparation may be used so that therapeutically effective amounts of the preparation are delivered into the bloodstream over many hours or days following transdermal injection or delivery.

The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is preferred particularly for buffers containing sodium ions.

The compounds useful in the methods and medicaments disclosed herein can be in either the acid (—OH) form or be N terminally amidated (—NH₂). The compounds can also be formulated as pharmaceutically acceptable salts (e.g., acid addition salts) and/or complexes thereof. Pharmaceutically acceptable salts are non-toxic salts at the concentration at which they are administered. The preparation of such salts can facilitate the pharmacological use by altering the physical-chemical characteristics of the composition without preventing the composition from exerting its physiological effect. Examples of useful alterations in physical properties include lowering the melting point to facilitate transmucosal administration and increasing the solubility to facilitate the administration of higher concentrations of the drug.

Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Carriers or excipients can also be used to facilitate administration of the compound. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents. The compositions or pharmaceutical composition can be administered by different routes including intravenously, intraperitoneal, subcutaneous, and intramuscular, orally, topically, transmucosally, or by pulmonary inhalation.

If desired, solutions of the above compositions may be thickened with a thickening agent such as methyl cellulose. They may be prepared in emulsified form, either water in oil or oil in water. Any of a wide variety of pharmaceutically acceptable emulsifying agents may be employed including, for example, acacia powder, a non-ionic surfactant (such as a Tween), or an ionic surfactant (such as alkali polyether alcohol sulfates or sulfonates, e.g. a Triton).

Compositions useful in the invention are prepared by mixing the ingredients following generally accepted procedures. For example, the selected components may be simply mixed in a blender or other standard device to produce a concentrated mixture which may then be adjusted to the final concentration and viscosity by the addition of water or thickening agent and possibly a buffer to control pH or an additional solute to control tonicity.

To assist in understanding the present methods, the following Examples are included. The experiments relating to the disclosed methods should not, of course, be construed as specifically limiting the invention and such variations of the invention, now known or later developed, which would be within the purview of one skilled in the art are considered to fall within the scope of the invention as described herein and hereinafter claimed.

### EXAMPLES

### Example 1

### Reduction of Food Intake by Endothelins and Endothelin Agonists

Materials & Methods: NIH/Swiss mice were fasted approximately 17 hours prior to initiation of the experiment. Peptides were injected intraperitoneally (i.p.) at time zero using doses as indicated. All mice received an intraperitoneal injection (200 μl) of either vehicle or compounds at doses indicated and were immediately presented with a pre-weighed food pellet. The food pellet was weighed at 30-minute, 1-hr, and 2-hr intervals after presentation to determine the amount of food eaten. Each point reflects n=4 for mice receiving an endothelin or endothelin agonist or n=3 for mice receiving vehicle. Compounds used in the experiment are listed in Table 5 below.

TABLE 5
Compounds

| | |
|---|---|
| Name | Sequence |

| | |
|---|---|
| Endothelin 1 | c(CSCSSLMDKECVYFCHLDIIW)-OH |
| | SEQ ID NO: 1 |
| Endothelin-3 | c(CTCFTYKDKECVYYCHLDIIW)-OH |
| | SEQ ID NO: 10 |
| Endothelin-2 | c(CSCSSWLDKECVYFCHLDIIW)-OH |
| | SEQ ID NO: 7 |
| [Succinyl(G1u⁹, Ala¹¹, Ala¹⁵]- | Succinyl-DEEAVYFAHLDIIW-OH |
| Endothelin-1-(8-21)] (IRL1620) | SEQ ID NO: 19 |
| Sarafotoxin S6c [Atractaspis | c(CTCNDMTDEECLNFCHQDVIW)-OH |
| engaddensis] | SEQ ID NO: 17 |

Food intake values reflect, at each time point, effect of test sample on food intake is expressed as % change relative to vehicle using the formula provided below.

% basal=−100*[1−(food intake of treatment group/food intake of vehicle group)].

Significant test sample effects were identified by ANOVA (p<0.05). Where a significant difference exists, test means were compared to the control mean using Dunnett's test. One-way ANOVA with Dunnett's post test was performed using GraphPad Prism® version 3.01 for Windows, GraphPad Software, San Diego, Calif. USA.

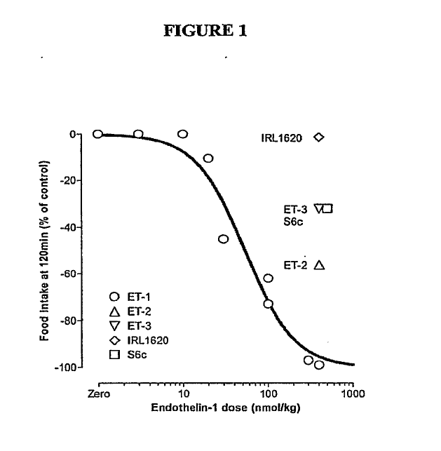

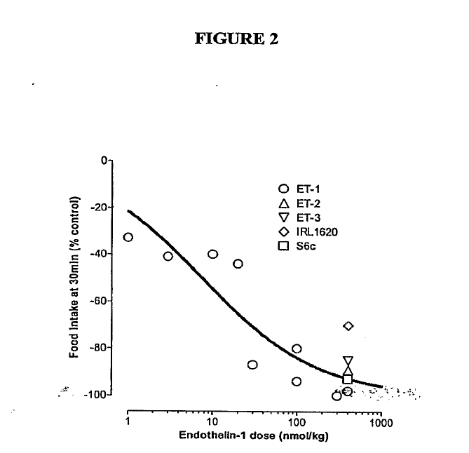

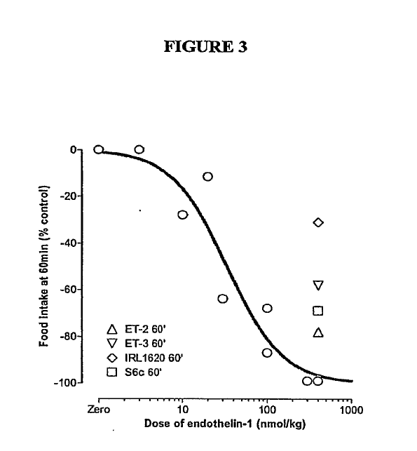

Results: There was a dose-dependent reduction in food intake following peripheral administration of ET-1 as well as with ET-2, ET-3 and Sarafotoxin S6c at all time points examined. See FIGS. 1, 2 and 3. The dose response for food intake reduction at 30 minutes indicated a similar potency for the agonists tested, while the food intake reduction at 60 minutes was intermediate. See FIGS. 2 and 3. IRL1620 reduced food intake at 30 and 60 minutes with no effect at 120 minutes, an effect likely due to a more rapid clearance/deactivation of the molecule. These data demonstrated that ET-1, ET-2, and ET-3 are potent and fully effective anorexigens in fasted mice without observable negative effects. The effectiveness of ET-3 in reducing food intake suggests that the anoretic effect is likely mediated by ET_{B} receptors since ET-3 binds ET_{A} poorly if at all.

### Example 2

### Reduction of Body Weight by Endothelins and Endothelin Agonists in DIO Mice

Materials & Methods: Diet induced obese (DIO) mice were employed. Obesity was induced by feeding a pelleted high-fat diet (58% of calories, #D12331, Research Diets, New Brunswick, N.J.) starting at 4 wk of age (20) for 6 wk (4 wk for the long-term study) prior to treatment. The mice then remained on this diet in powdered form throughout the treatment period unless otherwise noted. All animals were housed under a 12 hr:12 hr light-dark cycle at 21-23° C., and allowed ad libitum access to food pre- and post-treatment. Vehicle, ET-1 [SEQ ID NO: 1] (20 nmol/kg/d), or adenoregulin [SEQ ID NO: 33] (300 nmol/kg/d) were administered to DIO mice by Alzet® s.c. osmotic pumps. Mice were fed pelleted high-fat diet, and body weights and food intake were recorded weekly.

Body weight (BWt), at each time point, reflects the effect of test sample on BWt when expressed as % change relative to vehicle treated. For a treatment group:

% BWt loss=100*[(mean BWt change vehicle treated)−(mean BWt change treated)/mean BWt at day 0]

For an individual:

% BWt loss=100*[(mean BWt change vehicle treated)−(BWt change treated)/mean BWt at day 0].

Significant test sample effects were identified by ANOVA (p<0.05). Where a significant difference exists, test means were compared to the control mean using Dunnett's test. One-way ANOVA with Dunnett's post test was performed using GraphPad Prism® version 3.01 for Windows, GraphPad Software, San Diego Calif.

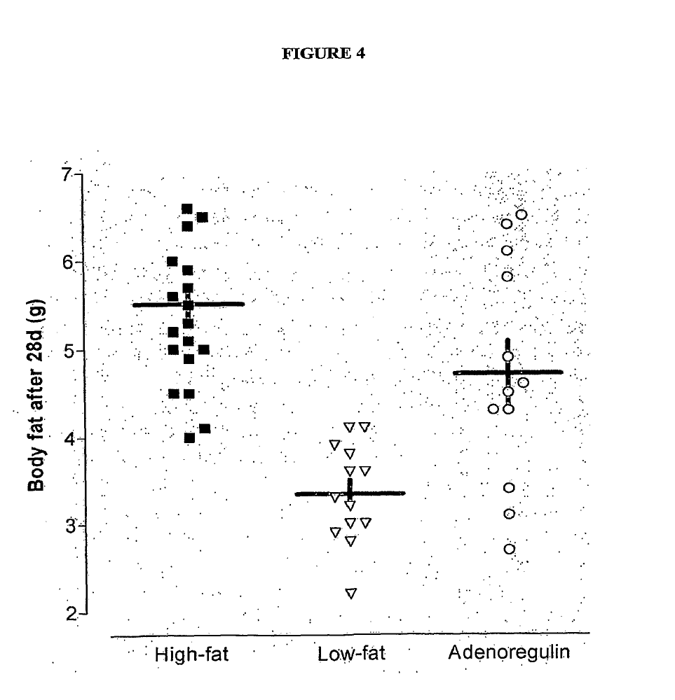

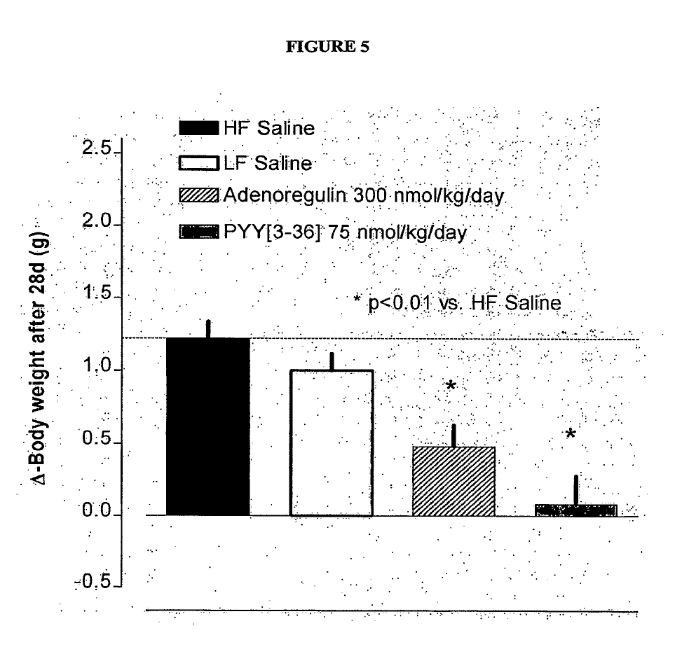

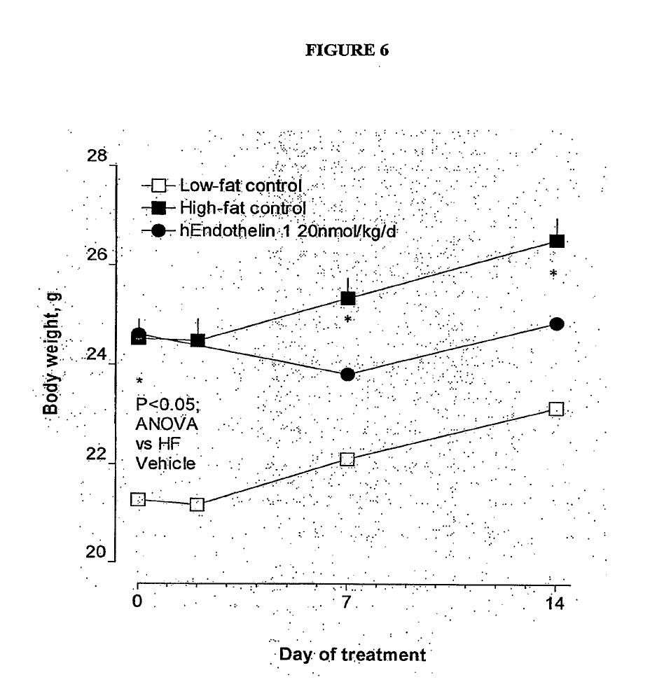

Results: The ET_{B} agonist, adenoregulin infused continuously for 2-4 weeks in DIO mice exhibited a sustained weight reducing effect (FIG. 5) as well as a reduction in body fat (FIG. 4). Notably, adenoregulin inhibited food intake in rodents at doses estimated to be less than those exerting antibiotic effects. Similarly, continuously infused ET-1 also reduced body weight in DIO mice versus controls (FIG. 6). This effect was attained without observable sickness behavior. In sum, these studies demonstrated the ability of ET-1 and an ET_{B} agonist to reduce food intake, resulting in a reduction in body weight and body fat.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1-74. (canceled)

75. A method to reduce body weight in a subject desirous or in need thereof comprising administering an endothelin or an endothelin agonist to a subject desirous or in need thereof in an amount effective to reduce body weight.

76. A method to reduce food intake in a subject desirous or in need thereof comprising administering an endothelin or an endothelin agonist to a subject desirous or in need thereof in an amount effective to reduce food intake.

77. A method to treat obesity in a subject desirous or in need thereof comprising administering an endothelin or an endothelin agonist to a subject desirous of in need thereof in an amount effective to treat obesity.

78. A method to prevent or treat a metabolic disorder in a subject desirous or in need thereof, comprising administering an endothelin or an endothelin agonist to a subject desirous or in need thereof in an amount effective to treat said metabolic disorder.

79. The method according to claim 78 wherein the metabolic disorder is obesity, diabetes mellitus, insulin-resistant syndrome, syndrome-X, or other hypernutrition disorders.

80. The method according to claim 75 wherein the subject is obese, overweight, desirous of reducing body weight or desirous of preventing a further increase in body weight associated with the disease or condition such as drug-induced weight gain.

81. The method according to claim 75 wherein said endothelin is at least one of endothelin-1, endothelin-2, or endothelin-3.

82. The method according to claim 81 wherein said endothelin is endothelin-1 or a sequence having at least 75% sequence identity to SEQ ID NO: 1.

83. The method according to claim 81 wherein said endothelin is endothelin-2 or a sequence having at least 75% sequence identity to SEQ ID NO: 7.

84. The method according to claim 81 wherein said endothelin is endothelin-3 or a sequence having at least 75% sequence identity to SEQ ID NO: 10.

85. The method according to claim 75 wherein said endothelin agonist is an analog of SEQ ID NO: 1, SEQ ID NO: 7, or SEQ ID NO: 10 containing not more than 5 amino acid substitutions, deletions or additions.

86. The method according to claim 85 wherein said analog contains not more that 5 amino acid substitutions.

87. The method according to claim 75 wherein
said endothelin is an endothelin-1 selected from at least one of the group consisting of SEQ ID NOs: 1-6; or
said endothelin is an endothelin-2 selected from at least one of the group consisting of SEQ ID NOs: 7-9; or
said endothelin is an endothelin-3 selected from at least one of the group consisting of SEQ ID NOs: 10-13.

88. The method according to claim 75 wherein said endothelin agonist is an endothelin analog containing not more than 35 amino acids.

89. The method according to claim 88 wherein said endothelin analog comprises a C terminal having the amino acid sequence FCHLDIIW.

90. The method according to claim 88 wherein said endothelin analog comprises a C terminal having the amino acid sequence FAHLDIIW.

91. The method according to claim 75 wherein said endothelin agonist is N-cis-2,6-dimethylpiperidinocarbonyl-L-γ-methylleucyl-D-1-methoxycarbonyltryptophanyl-D-norleucine.

92. The method according to claim 75 wherein said endothelin agonist is [Ala^{1,3,11,15}] endothelin-1 (SEQ ID NO: 14).

93. The method according to claim 75 wherein said endothelin agonist is a fragment of endothelin-1, endothelin-2, endothelin-3, wherein said fragment binds to and activates an ET_{A} or ET_{B} receptor.

94. The method according to claim 93 wherein said fragment comprises not more than 7 amino acid deletions.

95. The method according to claim 75 wherein the endothelin agonist contains not more than 10 amino acid substitutions, additions or deletions as compared to any one of SEQ ID NOs: 1, 7 and 10.

96. The method according to claim 93 wherein said endothelin agonist is (N-Succinyl-[Glu⁹, Ala^{11,15}]-Endothelin-1 fragment 8-21.

97. The method according to claim 95 wherein said endothelin agonist is (N-Acetyl-[Ala^{11,15}]-Endothelin-1 fragment 6-21.

98. The method according to claim 75 wherein said endothelin agonist is a sarafotoxin.

99. The method according to claim 98 wherein said sarafotoxin has an amino acid sequence selected from the group consisting of SEQ ID NO: 15, 16, 17, 18, 31 and 32.

100. The method according to claim 98 wherein said sarafotoxin has an amino acid sequence having at least 75% identity to SEQ ID NO: 15, 16, 17, 18, 31 and 32.

101. The method according to claim 98 wherein said sarafotoxin comprising a C terminal having the amino acid sequence FCHQDVIW.

102. The method according to claim 75 wherein said endothelin agonist is adenoregulin.

103. The method according to claim 102 wherein said adenoregulin has an amino acid sequence selected from the group comprising SEQ ID NO: 20-30.

104. The method according to claim 102 wherein said adenoregulin has an amino acid sequence having at least 75% identity to any one of SEQ ID NO: 20-30.

105. The method according to claim 75 wherein said endothelin or endothelin agonists is and ET_{B} receptor agonist, but not an ET_{A} receptor agonist.

106. The method according to claim 75 wherein said endothelin or endothelin agonist preferentially binds and activates an ET_{B} receptor compared to an ET_{A} receptor.

107. The method according to claim 75 further comprising administration of a second compound, wherein said compound induces satiety, reduces food intake, reduces or maintains body weight or any combination thereof.

108. The method according to claim 107 wherein said second compound is one of more of a compound selected from the group consisting of an exendin or agonist thereof, amylin or agonist analog thereof, PYY or agonist analogs thereof, leptin, oxyntomodulin, or a cholecystokinin (CCK).
